(19) United States
(12) Patent Application Publication
(10) Pub. No.: US 2026/0063612 A1
(43) Pub. Date: Mar. 5, 2026

(54) METHOD FOR PREDICTING CLAY MINERAL DEVELOPMENT IN LACUSTRINE SHALE BASED ON ARTIFICIAL INTELLIGENCE LEARNING OF LOGGING

(71) Applicant: Northeast Petroleum University, Daqing (CN)

(72) Inventors: Xiaofei FU, Daqing (CN); Xin LI, Daqing (CN); Bo LIU, Daqing (CN); Jinyou ZHANG, Daqing (CN); Yanfang LYU, Daqing (CN); Haixue WANG, Daqing (CN); Jun FENG, Daqing (CN); Lingdong MENG, Daqing (CN); Guidong PING, Daqing (CN)

(73) Assignee: Northeast Petroleum University, Daqing, HL (CN)

(21) Appl. No.: 19/379,600

(22) Filed: Nov. 4, 2025

(30) Foreign Application Priority Data
Dec. 19, 2024 (CN) 202411877062.0

(51) Int. Cl. G01N 33/24; G06N 3/0499; G06N 3/084

(52) U.S. Cl. CPC G01N 33/24; G06N 3/0499; G06N 3/084

(57) ABSTRACT

A method for predicting clay mineral development in lacustrine shale based on artificial intelligence is disclosed. The method comprises acquiring logging data of lacustrine shale, collecting rock samples of lacustrine shale, performing data preprocessing on the collected logging data, and acquiring types and contents of clay minerals in shale development a prediction model of clay mineral development is then developed based on an artificial intelligence. The artificial intelligence-based prediction model of clay mineral development is then optimized; and clay mineral development in lacustrine shale is predicted. In the present disclosure, the accuracy and efficiency of existing prediction methods are improved, allowing for the rapid acquisition of valuable information for the exploration and development of lacustrine shale.

### TECHNICAL FIELD

The present disclosure relates to the field of geological exploration technology, more particularly to a method for predicting clay mineral development in lacustrine shale based on artificial intelligence learning of logging.

### BACKGROUND

In the exploration and development of lacustrine shales, the development of clay minerals plays a crucial role in reservoir assessment and oil and gas extraction. Conventional clay mineral analysis methods typically rely on the rock thin-section observation and chemical analysis, which are not only time-consuming and labor-intensive but also difficult to apply for rapid and accurate predictions in large-scale exploration areas.

There are several methods for determining the types and contents of clay minerals using logging data, including the thorium-potassium crossplot method, the cation exchange capacity-hydrogen index crossplot method, and the stepwise multiple regression method. Although the thorium-potassium crossplot parameters are easy to collect, they can only be used for a qualitative evaluation of clay minerals. Accordingly, previous researchers proposed an improved thorium-potassium crossplot method, which can accurately determine the contents and types of clay minerals, but cannot accurately distinguish kaolinite and illite, which both exhibit high thorium content. The cation exchange capacity-hydrogen index crossplot method can distinguish smectite and illite, but it is limited in distinguishing chlorite from kaolinite. The stepwise multiple regression method has high interpretation accuracy, yet the selection of mathematical models is significantly interfered by human factors, making it difficult to ensure the optimal interpretation can be obtained.

With the continuous development of logging technology and the application of the artificial intelligence algorithm, a novel pathway is provided to predict the clay mineral development in lacustrine shale.

Accordingly, it is an urgent need for those skilled in the field to propose a method for predicting clay mineral development in lacustrine shale based on artificial intelligence learning of logging, thereby addressing the challenges existing in the prior art.

### SUMMARY

In view of the foregoing, the present disclosure provides a method for predicting a clay mineral development in lacustrine shale based on artificial intelligence learning of logging, which is used to solve the technical problems existing in the prior art.

In order to achieve the above objective, the present disclosure provides the following technical solution:
a method for predicting clay mineral development in lacustrine shale based on artificial intelligence learning of logging, the method includes the following steps:
acquiring logging data of lacustrine shale, and collecting rock samples of lacustrine shale;
performing a data preprocessing on the collected logging data of lacustrine shale;
acquiring types and contents of clay minerals in shale development by testing the rock samples of lacustrine shale;
according to the preprocessed logging data of lacustrine shale and the types and contents of clay minerals in shale development, constructing a artificial intelligence-based prediction model of clay mineral development;
optimizing the artificial intelligence-based prediction model of clay mineral development; and
predicting the clay mineral development in lacustrine shale through the optimized artificial intelligence-based prediction model of clay mineral development.

In the above method, optionally, the logging data of lacustrine shale includes: density, natural potential, natural gamma, acoustic time difference, borehole diameter, deep lateral resistivity, shallow lateral resistivity, and porosity.

In the above method, optionally, the specific content of collecting the rock samples of the lacustrine shale is as follows:
in a shale exploration area, selecting shale cores for the shale sample collection by observing distribution characteristics of shale.

In the above method, optionally, the specific content of acquiring the types and contents of clay minerals in shale development by testing the rock samples of lacustrine shale is as follows:
through an X-ray diffraction (XRD) shale mineral type test and an XRD clay mineral content test, acquiring the types and contents of clay minerals in shale development.

In the above method, optionally, the specific content of constructing the artificial intelligence-based prediction model of clay mineral development is as follows:
dividing the preprocessed logging data of lacustrine shale and the types and contents of clay minerals in shale development into a training set and a test set, and inputting the training set into a back-propagation (BP) neural network model for training, during the training process, adjusting the BP neural network model by a back-propagation algorithm.

In the above method, optionally, the specific content of the back propagation algorithm is as follows:
including one input layer, a plurality of hidden layers and one output layer, and the formula for forward propagation is as follows:

$${z}_{j} = {\sum }_{i = 1}^{M} {w}_{ij} {x}_{i} + {b}_{j}$$

$${x}_{j} = f ( {z}_{j} ) = \frac{1}{1 + {e}^{- {z}_{j}}}$$

where zⱼ is an net output value of a j^{th} node, wᵢⱼ is a weight value between a i^{th} node and the j^{th} node, xᵢ is an input value of the i^{th} node, bⱼ is a threshold value of the j^{th} node, M is a number of input layer nodes, xⱼ is an output value of the j^{th} node, ƒ(zⱼ) is a sigmoid activation function;
the formula for backward propagation is:

$$E = \frac{1}{2} {\sum }_{j = 1}^{N} {x}_{j} - {y}_{j}$$

where E is a loss function, N is a number of output layer nodes, and yⱼ is a label value of the j^{th} node.

In the above method, optionally, the specific content of predicting the clay mineral development in lacustrine shale through the optimized artificial intelligence-based prediction model of clay mineral development is as follows:
by comparing prediction results with the actual observation values, evaluating the accuracy and reliability of the artificial intelligence-based prediction model of clay mineral development, if the prediction results are undesirable, returning to the optimization steps of the artificial intelligence-based prediction model of clay mineral development for adjustment.

According to the above technical solution, compared with the prior art, the present disclosure discloses a method for predicting clay mineral development in lacustrine shale based on artificial intelligence learning of logging, which has the following beneficial effects:
1) the accuracy and efficiency of prediction are improved, allowing for the rapid availability of valuable information for the exploration and development of lacustrine shale;
2) the cost and time consumption of conventional methods are reduced, and the complicated laboratory analysis process is avoided;
3) based on a large amount of actual logging data and an advanced artificial intelligence algorithm, featuring strong generalization ability and adaptability.

### BRIEF DESCRIPTION OF THE DRAWINGS

To explain the embodiments of the present disclosure or the technical solutions in the prior art more clearly, a brief introduction will be made to the accompanying drawings used in the embodiments or the description of the prior art. It is obvious that the drawings in the description below are only some embodiments of the present disclosure, and those ordinarily skilled in the art can obtain other drawings according to these drawings without creative work.

FIG. 1 is a flowchart of a method for predicting a clay mineral development in lacustrine shale based on artificial intelligence learning of logging provided by the present disclosure;

FIG. 2 is a clay mineral development prediction diagram provided by an embodiment of the present disclosure.

### DETAILED DESCRIPTION OF THE EMBODIMENTS

The following description will clearly and completely explain the technical solutions of the embodiments of the present disclosure with reference to the accompanying drawings. Apparently, the described embodiments are only some but not all of the embodiments of the present disclosure. All other embodiments obtained by those of ordinary skill in the art based on the embodiments of the present disclosure without involving any creative effort shall fall within the scope of protection of the present disclosure.

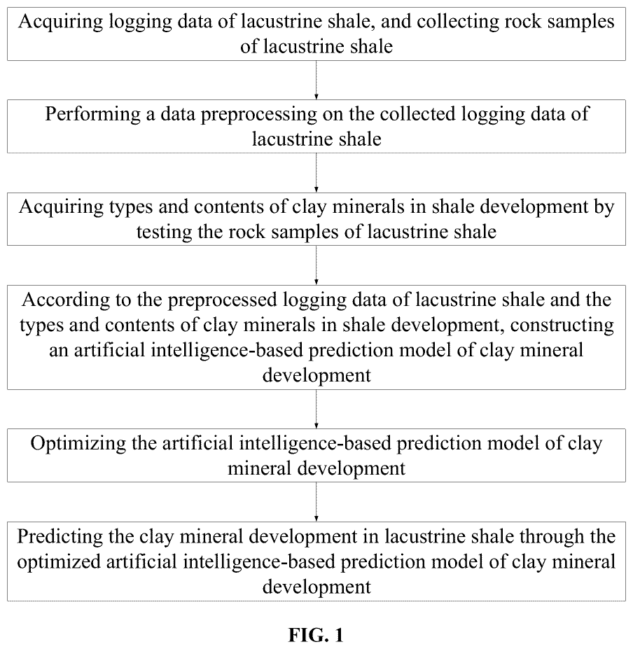

With reference to FIG. 1, a method for predicting a clay mineral development in lacustrine shale based on artificial intelligence learning of logging, including the following steps:
the logging data of lacustrine shale is acquired, and the rock samples of lacustrine shale are collected;
the data preprocessing is performed on the collected logging data of lacustrine shale;
specifically, the collected logging data are cleaned, which involves the removal of outliers and noises, and the data standardization and normalization are performed, thereby improving the quality and availability of the data;
the types and contents of clay minerals in shale development are acquired by testing the rock samples of lacustrine shale;
according to the preprocessed logging data of lacustrine shale and the types and contents of clay minerals in shale development, the artificial intelligence-based prediction model of clay mineral development is constructed;
the artificial intelligence-based prediction model of clay mineral development is optimized; and
the clay mineral development in lacustrine shale is predicted through the optimized artificial intelligence-based prediction model of clay mineral development.

Further, the logging data of lacustrine shale includes: density, natural potential, natural gamma, acoustic time difference, borehole diameter, deep lateral resistivity, shallow lateral resistivity, and porosity.

Further, the specific content of collecting the rock samples of the lacustrine shale is as follows:
in a shale exploration area, the shale cores are selected for the shale sample collection by observing the distribution characteristics of the shale.

Further, the specific content of acquiring the types and contents of clay minerals in shale development by testing the rock samples of lacustrine shale is as follows:
through the XRD shale mineral type test and the XRD clay mineral content test, the types and contents of clay minerals in shale development are acquired.

XRD is a technique for material structure analysis that utilizes the diffraction effect of X-rays by crystalline substances. When X-rays are projected into a crystal, they are scattered by the atoms in the crystal. These scattered waves resemble spherical waves emitting from the center of the atom. Since the periodic arrangement of atoms in the crystal, there is a fixed phase relationship between these scattered spherical waves. This results in the waves reinforcing each other in certain scattering directions and canceling each other out in certain directions, thereby giving rise to the diffraction phenomenon.

Through the analysis of clay minerals by XRD technology, the accurate identification results of mineral types can be obtained. For example, kaolinite, smectite, and illite are common types of clay minerals, and their X-ray diffraction patterns exhibit characteristic peak positions and peak shapes. Through the analysis of the experimental data, the different types of clay minerals can be clearly distinguished.

Further, the specific content of constructing the artificial intelligence-based prediction model of clay mineral development is as follows:
the preprocessed logging data of lacustrine shale and the types and contents of clay minerals in shale development are divided into the training set and the test set, and the training set is input into the BP neural network model for training. During the training process, the BP neural network model is adjusted by the back propagation algorithm.

Further, the specific content of the back propagation algorithm is as follows:
one input layer, a plurality of hidden layers and one output layer are included, and the formula for forward propagation is as follows:

$${z}_{j} = {\sum }_{i = 1}^{M} {w}_{ij} {x}_{i} + {b}_{j}$$

$${x}_{j} = f ( {z}_{j} ) = \frac{1}{1 + {e}^{- {z}_{j}}}$$

where zⱼ is the net output value of the j^{th} node, wᵢⱼ is the weight value between the i^{th} node and the j^{th} node, xᵢ is the input value of the i^{th} node, bⱼ is the threshold value of the j^{th} node, M is the number of input layer nodes, xⱼ is the output value of the j^{th} node, ƒ(zⱼ) is the sigmoid activation function;
the formula for backward propagation is:

$$E = \frac{1}{2} {\sum }_{j = 1}^{N} {x}_{j} - {y}_{j}$$

where E is the loss function, N is the number of output layer nodes, and yⱼ is the label value of the j^{th} node.

Specifically, cross-validation is employed to avoid the situation that the construction model may not be an optimal solution caused by a single random selection of data, thereby enhancing the generalization capability of the prediction model.

Further, the specific content of predicting the clay mineral development in lacustrine shale through the optimized artificial intelligence-based prediction model of clay mineral development is as follows:
by comparing prediction results with the actual observation values, the accuracy and reliability of the artificial intelligence-based prediction model of clay mineral development are evaluated. If the prediction results are undesirable, return to the optimization steps of the artificial intelligence-based prediction model of clay mineral development for adjustment.

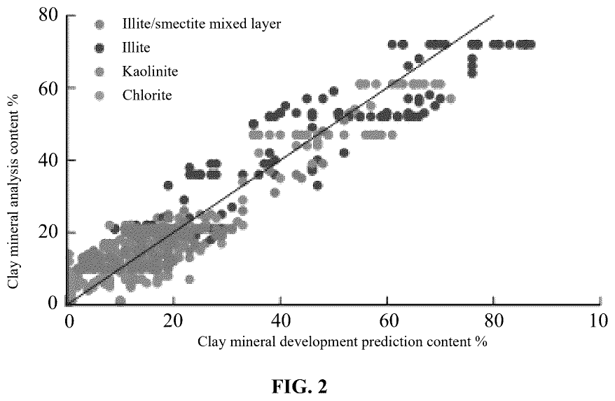

In a specific embodiment, the logging data of lacustrine shale in a certain area is acquired, and the rock samples of lacustrine shale is collected; the data preprocessing is performed on the collected logging data of lacustrine shale; the types and contents of clay minerals in shale development are acquired by testing the rock samples of lacustrine shale; according to the preprocessed logging data of lacustrine shale and the types and contents of clay minerals in shale development, the artificial intelligence-based prediction model of clay mineral development is constructed; the artificial intelligence-based prediction model of clay mineral development is optimized; and the clay mineral development in lacustrine shale is predicted through the optimized artificial intelligence-based prediction model of clay mineral development. The predicted content distribution diagrams for illite/smectite mixed layer, illite, kaolinite, and chlorite are shown in FIG. 2.

The embodiments in this specification are described in a progressive manner. Each embodiment primarily highlights the differences from other embodiments, and identical or similar aspects among the embodiments should be cross-referenced as necessary.

The aforementioned description of the disclosed embodiments enables those skilled in the art to practice or use the present disclosure. Various modifications to these embodiments will be apparent to those skilled in the art. The general principles defined herein may be implemented in other embodiments without departing from the spirit or scope of the present disclosure. Therefore, the present disclosure is not to be limited to the embodiments shown herein, but rather to the broadest scope consistent with the principles and novel features disclosed herein.

What is claimed is:

1. A method for predicting clay mineral development in lacustrine shale based on artificial intelligence, comprising the following steps:
acquiring logging data of lacustrine shale, and collecting rock samples of lacustrine shale;
performing data preprocessing on the logging data of the lacustrine shale;
acquiring types and contents of clay minerals by testing the rock samples of the lacustrine shale;
according to the preprocessed logging data and the types and contents of clay minerals in the lacustrine shale, constructing an artificial intelligence-based prediction model of clay mineral development;
optimizing the artificial intelligence-based prediction model of clay mineral development; and
predicting clay mineral development in lacustrine shale through the optimized artificial intelligence-based prediction model of clay mineral development.

2. The method for predicting the clay mineral development in lacustrine shale based on artificial intelligence according to claim 1, wherein the logging data of lacustrine shale comprises: density, natural potential, natural gamma, acoustic time difference, borehole diameter, deep lateral resistivity, shallow lateral resistivity, and porosity.

3. The method for predicting the clay mineral development in lacustrine shale based on artificial intelligence according to claim 1, wherein the collecting the rock samples of the lacustrine shale comprises:
in a shale exploration area, selecting shale cores for the shale sample collection by observing distribution characteristics of shale.

4. The method for predicting the clay mineral development in lacustrine shale based on artificial intelligence according to claim 1, wherein acquiring the types and contents of clay minerals in shale development by testing the rock samples of the lacustrine shale comprises:
through an XRD shale mineral type test and an XRD clay mineral content test, acquiring the types and contents of clay minerals in shale development.

5. The method for predicting the clay mineral development in lacustrine shale based on artificial intelligence according to claim 1, wherein constructing the prediction model of clay mineral development based on the artificial intelligence comprises:
dividing the preprocessed logging data of lacustrine shale and the types and contents of clay minerals in the lacustrine shale into a training set and a test set, and inputting the training set into a BP neural network model for training, and, during the training process, adjusting the BP neural network model by a back propagation algorithm.

6. The method for predicting the clay mineral development in lacustrine shale based on artificial intelligence according to claim 5, wherein the specific content of the back propagation algorithm is as follows:
the formula for forward propagation comprises one input layer, a plurality of hidden layers and one output layer, according to the formulae as follows:
$${z}_{j} = {{\sum }_{}}_{i = 1}^{M} {w}_{ij} {x}_{i} + {b}_{j}$$
$${x}_{j} = f ( {z}_{j} ) = \frac{1}{1 + {e}^{- {z}_{j}}}$$
where zⱼ is an net output value of a j^{th} node, wᵢⱼ is a weight value between a i^{th} node and the j^{th} node, xᵢ is an input value of the i^{th} node, bⱼ is a threshold value of the j^{th} node, M is a number of input layer nodes, xⱼ is an output value of the j^{th} node, ƒ(zⱼ) is a sigmoid activation function;
and wherein the formula for backward propagation is:
$$E = \frac{1}{2} {{\sum }_{}}_{j = 1}^{N} {x}_{j} - {y}_{j}$$
where E is a loss function, N is a number of output layer nodes, and yⱼ is a label value of the j^{th} node.

7. The method for predicting the clay mineral development in lacustrine shale based on artificial intelligence according to claim 1, wherein predicting the clay mineral development in lacustrine shale through the optimized artificial intelligence-based prediction model of clay mineral development is as follows:
by comparing prediction results with actual observation values, evaluating accuracy and reliability of the prediction model of clay mineral development based on the artificial intelligence, and if the prediction results are undesirable, returning to the optimization steps of the prediction model of clay mineral development based on the artificial intelligence for adjustment.
